(19) United States
(12) Patent Application Publication
(10) Pub. No.: US 2025/0127949 A1
(43) Pub. Date: Apr. 24, 2025

(54) AEROSOL SPRAYS, METHODS OF GENERATING AEROSOL SPRAYS, AND AEROSOL DISPENSING SYSTEMS

(71) Applicant: Medspray B.V., Enschede (NL)

(72) Inventors: Anne Mechteld Reuver, Enschede (NL); Wietze Nijdam, Enschede (NL); Franciscus Martinus Verhoeven, Enschede (NL); Randy Pumell Washington, Lake Villa, IL (US); Therese A Rozek, Union Grove, WI (US); Keegen Burggren, Mount Pleasant, WI (US); Kenneth Michaels, Spring Grove, IL (US); Sebastian Hasik, Antioch, IL (US); Dallas Matz, Racine, WI (US); Alex Hooper, Milwaukee, WI (US)

(21) Appl. No.: 18/272,663

(22) PCT Filed: Jan. 17, 2022
(86) PCT No.: PCT/IB2022/050356

§ 371 Date: Jul. 17, 2023

Related U.S. Application Data
(60) Provisional application No. 63/138,505, filed on Jan. 17, 2021.

(51) Int. Cl. A61L 9/14; A61L 9/012; B05B 1/18

(52) U.S. Cl. CPC A61L 9/14; A61L 9/012; B05B 1/18; A61L 2202/11

(57) ABSTRACT

Aerosol sprays, methods of generating aerosol sprays, and devices for dispensing aerosol sprays. The aerosol sprays have properties that are highly desirable for products such as air fresheners, including sizes of particles in the sprays and span factors of the particles. The methods and systems that provide the aerosol sprays use a membrane having micropores that forms Rayleigh jets that subsequently break up into the aerosol particles. The systems used to generate the aerosol sprays have a non-pressurized container for the product to be dispensed and a pump to provide the force to push the product through the micropores of the membrane.

### BACKGROUND

### Field of the Invention

The invention relates to aerosol sprays, methods of generating aerosol sprays, and systems for dispensing aerosol sprays.

### Related Art

Aerosol sprays are used to provide many consumer products, including air fresheners, deodorizers, disinfectants, insecticides, and cleaners. To provide such aerosol sprays, different types of dispensing systems have been developed. Some aerosol spray dispensing systems have or are connected to a power source causing the system to automatically dispense the sprays. Other types of aerosol dispensing systems are provided in containers that are actuated by a user on demand.

Even with the wide variety of dispensing systems and dispensing system configurations, for some products it can be difficult to generate an aerosol spray having desired properties. For example, in the case of air freshening products, the goal is for the system to provide the product such that a sufficient amount of fragrance experience is achieved soon after the dispensing, but also such that there is longevity in the fragrance experience. To achieve this, it is often important that the size of the particles in the aerosol spray be in a certain range, and that the particles do not greatly deviate from certain sizes. It is also often important that particles in, for example, an air freshening spray do not fall to the ground too quickly after the spray is dispensed and that the particles are discharged a sufficient distance from the dispensing system.

Many aerosol dispensing systems include a container that holds a product with liquid and gas parts. The gas included with the liquid product acts as a propellant to discharge the liquid product from the container when the system is actuated. The propellant pressurizes the container holding the liquid composition, and provides a force to expel the liquid composition from the container when the system is actuated. For such systems, there are two main types of propellants: (1) liquefied gas propellants (LPGs), such as hydrocarbon and hydrofluorocarbon (HFC) propellants, and (2) compressed gas propellants (CGAs), such as carbon dioxide and nitrogen. Generally speaking, as compared to CGA propellant systems, aerosol dispensing systems that use LPG propellants are able to produce smaller, more consistent sized particles in sprays. Thus, from a performance standpoint, systems using LPG propellants are often superior to systems using CGAs as propellants. But, LPG propellants include a high amount of volatile organic compounds (VOCs), thereby making their use subject to various regulations.

### SUMMARY OF THE INVENTION

The present invention relates to aerosol sprays, methods of generating aerosol sprays, and systems for dispensing aerosol sprays. An aerosol spray is a suspension of particles (solid or liquid) in air or a gas. In many aerosol sprays, the spray is dispensed from a system with the use of a propellant gas. As discussed below, sprays according to embodiments of the invention are not formed using propellant gases. Thus, as used herein, an aerosol spray may mean a collection of particles suspended in normal air without a further propellant gas.

The aerosol sprays described herein can be formulated to provide many different types of products. In some embodiments of the invention, the aerosol spray includes a fragrance compound(s) to provide, for example, an air freshening product. Examples of other types of products that can be provided with the aerosol sprays described herein are an insect repellant, a deodorizing or malodor control substance, a surface cleaning substance, a carpet cleaning substance, a window cleaner, and many other types of products.

According to one aspect, our invention provides an aerosol spray dispensing system. The system includes a non-pressurized container that contains a liquid composition. The system also includes a spray nozzle in fluid communication with the container, the spray nozzle including a membrane having micropores through which the product passes as the product is dispensed from the system. The system further includes a pump configured to provide a force causing the liquid formulation to move from the container and through the spray nozzle such that the liquid formulation is discharged from the system as an aerosol spray.

The micropores comprise a first group of at least one micropore adjacent a second group of micropores. Said first group releases a first aerosol spray portion and said second group releases a second aerosol spray portion, adjacent the first aerosol portion. A spray density of the first aerosol portion emanating from the first group, according to the invention, is larger than a spray density of the second aerosol portion emanating from the second group. The spray density is being defined as the spray flux, i.e. the spray volume per square centimetre, in a plane perpendicular to the axis of the nozzle, i.e. the principal direction of propagation of the spray.

The higher spray density within the first group creates a substantial drag adjacent the spray portion that is released by the second group of micropores. The spray jets emanating from the second group benefit from the slip stream caused by the first group to reach a greater spray distance and less fallout. The relatively high spray density in the first aerosol portion may even lead to mutual coalescence of the droplets within this portion of the spray that even enhances this mechanism.

The higher aerosol density within the first aerosol portion may be created by a larger pore size or a larger packing density of the micropore(s) within the first group as compared to the second group. In an embodiment of the invention, the first group comprises micropores having a larger pore density than a pore density of the micropores within the second group of micropores. In an embodiment of the invention, the first group comprises micropores having a larger average size than an average size of the micropores within the second group of micropores.

A different aerosol density may alternatively be realized by means of a spray angle of the subject aerosol portion. In an embodiment of the invention, the micropores within the second group of micropores release a microjet under a diverging jet angle with respect to an axis of the spray nozzle. In an embodiment of the invention, the at least one micropore within the first group releases a microjet substantially parallel to the axis of the spray nozzle.

In an embodiment of the invention, the second group of micropores surrounds the first group. In an embodiment of the invention, the first group and the second group each comprise at least one ring of micropores, said rings of micropores being substantially con-centric with one another. Particles in the spray particularly have a Dv(50) size of about 30 μm to about 70 μm.

According to another aspect, the invention provides a method of generating an aerosol spray. The method includes steps of forcing a liquid composition out of a non-pressurized container, and passing the liquid composition through micropores on a membrane such that Rayleigh jets are produced, with the Rayleigh jets subsequently breaking up into particles of the aerosol spray. The liquid compound may include one or more of an air freshening composition including (i) fragrance oil and (ii) water or a solvent; a deodorizer agent; a disinfectant agent; an insecticide agent; and a cleaning agent. The liquid composition particularly includes (i) fragrance oil and (ii) water or a solvent.

According to yet another aspect, our invention provides an aerosol spray with particles of a liquid composition. The particles have a Dv(50) particle size of about 30 μm to about 70 μm, and the aerosol spray has a spray experience factor with a (negative) spray efficacy of about up to about −3300 and a (negative) span factor of up to about −1.25. The aerosol comprises a first aerosol portion adjacent an second aerosol portion, wherein said first aerosol portion has a larger spray density than said second aerosol portion. The liquid composition particularly includes a fragrance oil, a deodorizer agent, a disinfectant agent, an insecticide agent, or a cleaning agent.

According to a further aspect, the invention provides an aerosol spray with particles of a liquid composition that includes a fragrance oil. The particles have a Dv(50) particle size of about 30 μm to about 70 μm, and the aerosol spray has a particle quality factor with a (negative) Dv(90) particle size of up to about −90 μm and a (negative) span factor of up to about −1.25.

Particular properties of the aerosol sprays described herein make the sprays useful for a wide variety of products. One such property is the size of the particles in the spray. The particle size may be characterized by the Dv(50) of the particles, which is the diameter for which 50% of the total spray volume is made up of droplets of equal or lesser diameter. In some embodiments of the invention wherein the aerosol spray is, for example, an air freshening product, the particles range in Dv(50) size from about 30 μm to about 70 μm. In more preferred embodiments of the invention, the particles range in Dv(50) size from about 35 μm to about 45 μm. The sizes of particles in aerosol sprays generated by the methods and systems described herein will be further described below and compared to sprays generated by other types of systems.

Another advantageous property of aerosol sprays according to embodiments of the invention is the distribution of the sizes of the particles in the sprays. The size distribution can be quantified as the span factor, which is defined by the following equation:

$$Span Factor = \frac{Dv ( 90 ) - Dv ( 10 )}{Dv ( 50 )}$$

where Dv(10) is the diameter for which 10% of the total spray volume is made of droplets of equal or lesser diameter, Dv(50) is the diameter for which 50% of the total spray volume is made of droplets of equal or lesser diameter, and Dv(90) is the diameter for which 90% of the total spray volume is made of droplets of equal or lesser diameter. In embodiments of the invention, the span factor may range from about 0.75to about 1.25. In particular embodiments of the invention using, particularly, air freshening compounds with a water formulation, the span factor for the particles may range from about 0.75 to about 1.0. In other particular embodiments of the invention using liquid compounds in a solvent-based formulation, particularly air freshening compounds, the span factor for the particles in the aerosol spray may range from about 0.80 to about 1.1.

Still other advantageous properties of aerosol sprays according to embodiments of the invention are the distance spray particles travel from the dispensing system, the low amount of fallout of the particles from the spray in the air onto the ground, and the longevity of spray particles in the air over time. Methods for determining these properties will be described in conjunction with the comparative experiments described below.

Also described below are combinations of the properties of the aerosol sprays, such as a spray experience factor. As used herein, the spray experience factor is defined herein by the combination of spray efficacy and the negative of the span factor of the particles, where spray efficacy is defined as the negative of the product of the percent fallout and spray distance. Those skilled in the art will appreciate that the spray experience factor is indicative of the performance of an aerosol spray in a product such as an air freshener. In embodiments of the invention the spray experience factor has a (negative) spray efficacy of about 0 up to about −3300 and a (negative) span factor of up to about −1.25.In particular embodiments of the invention providing liquid sprays with a water-based formulation, particularly air freshening sprays, the spray experience factor has a spray efficacy of about 0 to about −400 and a (negative) span factor of about −0.75 to about −1.0. In other particular embodiments of the invention providing liquid sprays with a solvent-based formulation, particularly air freshening sprays, the spray experience factor has a spray efficacy of about −950 to about −3300 and a (negative) span factor of about −0.80 to about −1.1. The comparative data discussed below demonstrates that fragrant sprays dispensed from prior art systems do not have spray experience factors falling in the range of the spray experience factors of aerosol sprays according to embodiments of the invention.

Another combination of properties of sprays according to embodiments of the invention that is indicative of performance is a particle quality factor, which is defined herein by the negative Dv(90) particle size for the spray and the negative of the span factor for the spray particles. As will be appreciated by those skilled in the art, Dv(90) particle size is indicative of the amount of larger size particles in a spray, e.g., a lower Dv(90) indicates a lower amount of large particles. And, a lower number of large particles usually equates to less fallout of the spray. In embodiments of the invention, the particle quality factor has a (negative) Dv(90) of up to about −90 μm and a (negative) span factor of up to about −1.25. In particular embodiments of the invention providing liquid sprays with a water-based formulation, particularly air freshening sprays, the particle quality factor has a (negative) Dv(90) of about −50 μm to about −65 μm and a (negative) span factor of about −0.75 to about −1.0. In other particular embodiments of the invention providing liquid sprays with a solvent-based formulation, particularly air freshening sprays, the particle quality factor has a (negative) Dv(90) of about −40 μm to about −65 μm and a (negative) span factor of about −0.8 to about −1.1. The comparative data discussed below demonstrates that fragrant sprays dispensed from prior art systems do not have particle quality factors falling in the range of the particle quality factors of aerosol sprays according to embodiments of the invention.

### BRIEF DESCRIPTION OF THE DRAWINGS

FIG. 1 is a view of a spray nozzle according to embodiments of the invention.

FIG. 2 is a view of a membrane with micropores in the spray nozzle shown in FIG. 1.

FIG. 3 show the cone angle for an aerosol spray emanating from a nozzle according to an embodiment of the invention.

FIG. 4 is a view of a membrane with micropores according to another embodiment of the invention.

FIG. 5 is a view of an automatic aerosol spray dispensing system according to an embodiment of the invention.

FIG. 6 is another view of the automatic aerosol spray dispensing system shown in FIG. 5.

FIG. 7 is a view of a base container aerosol spray dispensing system according to an embodiment of the invention.

FIG. 8 is a cross-sectional view of the base container aerosol system shown in FIG. 7.

FIGS. 9A-9C show the particle sizes of aerosol sprays according to embodiments of the invention in comparison to the particle sizes of aerosol sprays generated by comparison systems.

FIG. 10 shows the span factors of aerosol sprays according to embodiments of the invention in comparison to the span factors of aerosol sprays generated by comparison systems.

FIG. 11 shows the spray distances of aerosol sprays dispensed from a system FIG. 11 according to embodiments of the invention in comparison to the spray distances of aerosol sprays from comparison systems.

FIG. 12 shows the fallout of aerosol sprays according to embodiments of the FIG. 12 invention in comparison to the fallout of aerosol sprays generated by comparison systems.

FIG. 13 shows the fallout of aerosol sprays generated using different spray FIG. 13 nozzles according to embodiments of the invention.

FIG. 14 shows the longevity of aerosol sprays according to embodiments of the FIG. 14 invention in comparison to the longevity of aerosol sprays generated by comparison systems.

FIG. 15 shows the spray experience factors of aerosol sprays according to embodiments of the invention in comparison to the spray experience factors aerosol sprays generated by comparison systems.

FIG. 16 shows the particle quality factors of aerosol sprays according to embodiments of the invention in comparison to the particle quality factors of aerosol sprays generated by comparison systems.

FIG. 17 shows a particle size distribution of a liquid spray by a conventional nozzle.

FIG. 18 shows a particle size distribution within a liquid spray according to the invention.

### DETAILED DESCRIPTION OF THE INVENTION

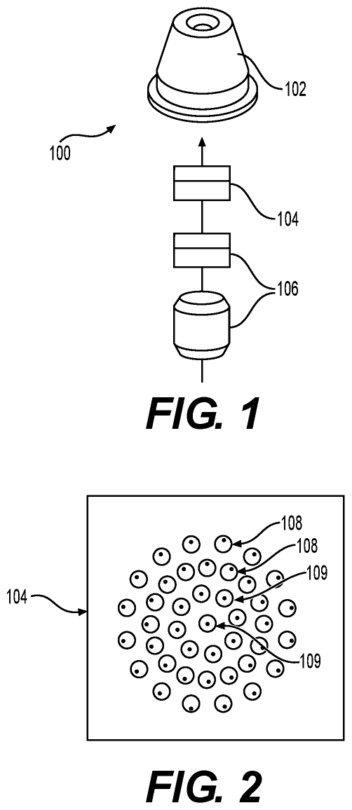

Aerosol sprays as described herein may be generated using an aerosol dispensing system that includes a spray nozzle through which the product forming the aerosol spray passes. A spray nozzle 100 according to embodiments of the invention is shown in FIGS. 1-3. The spray nozzle 100 could be used, for example, in automatic aerosol dispensing systems, as will be described below. The spray nozzle 100 includes a plastic cap structure 102, a membrane 104, and filters 106 for catching large particles before the particles reach the membrane 104.

It should be noted that nozzles according to embodiments of the invention are not limited to the configuration of the spray nozzle 100 depicted in FIG. 1. For example, in other embodiments filtering is performed before the nozzle structure. Thus, some spray nozzles do not include the filters 105 and 106. In particular embodiments of the invention, the membranes used with the nozzles are silicon wafer chips that are created using well-known manufacturing techniques that are often used to product semiconductors. Examples of such silicon wafer chips and their manufacture can be seen in U.S. Pat. Nos. 8,936,160, 8,814,059, 9,566,398, and 10,632,265, which are hereby incorporated by reference their entirety.

As shown in FIG. 2, the membrane 104 includes a plurality of micropores 108 that are arranged in two concentric outer circles to form a second group of micropores. A first group of micropores 109 is provided at the centre of the outer circles in the form of a concentric inner circle and a single micropore 109 in the middle. The corresponding dispensing system in which the spray nozzle 100 is used is configured such that the product to be dispensed travels through the micropores in the membrane.

As a result of the product passing through the micropores in the membrane, the product emanates from the nozzle 100 in Rayleigh jets that subsequently break up into the aerosol particles making up the spray. Rayleigh jests are a phenomenon that occurs when discharging a liquid from a nozzle at a large velocity such that a continuous jet is formed. And, due to capillary forces, the Rayleigh jets break up into droplets soon after exiting the nozzle. With methods and systems described herein, the result is an aerosol spray having many outstanding properties.

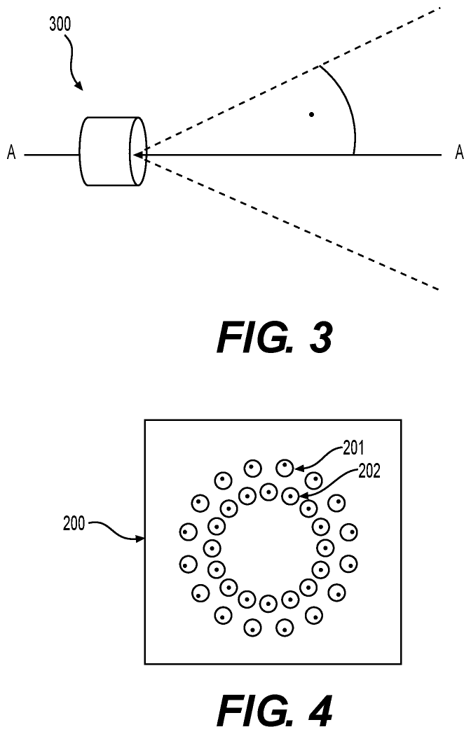

As indicated by the eccentric dots in the micropores 108 of the second group of micropores that is distributed over both outer concentric rings, these micropores 108 release a spray jet under an angle α, see FIG. 3, with respect to the centerline of the subject pore. These micropores are oriented such that the resulting spray will feature a corresponding cone angle a with respect to the centerline of the spray nozzle. The micropores 109 within the first group that is in the centre, however, are shown with a dot in their middle to indicate that these micropores release a spray jet along their centerline, i.e. with substantially no cone angle α.

The cone angle a results in a diverging spray pattern and correspondingly decreasing spray density within this part 108 of the spray. The central pores, however, are packed more closely together and release their spray jets closer to one another, without substantially no widening or less widening. This creates a substantial drag in de middle of the spray, caused by the relatively high spray density of this first group of micropores 109. The spray jets emanating from the surrounding micropores 108 benefit from the slip stream caused by the central group and will reach a greater spray distance and experience less fallout than without this central croup 109. The relatively high spray density in the inner group 109 may even lead to mutual coalescence of the droplets with the spray jets of this inner group that even enhances this mechanism.

It should be noted that nozzles according to embodiments of the invention are not limited to the configuration of the spray nozzle 100 depicted in FIG. 2. Particularly, the layout and the number of the micropores in the membranes in spray nozzles according to embodiments of the invention is not limited to the configuration shown in FIG. 1. In other embodiments, the micropores of the spray nozzles need not be positioned in circles. For example, in other embodiments, the micropores may be laid out in other symmetrical geometries, such as a square or star shape. Still further, in other embodiments of the invention, multiple membranes may be used in one nozzle structure, with the micropores in each of the multiple membranes providing parts of the total output spray from the nozzle.

For example, FIG. 4 shows a view of another spray nozzle 200. In this embodiment, the nozzle 200 includes an inner concentric circle of micropores 202, surrounded by an outer concentric circle of micropores 202. The micropores of the outer circle 201 each release a spray jet under an inclined angle towards the centerline of the respective micropore to create a diverging spray cone with a cone angle a. The micropores of the inner circle 202 release spray jets with no or a lesser angle towards the centerline to create a spray portion with a higher spray density that provides a slip stream for the cone spray portion. In other embodiments, the micropores of the spray nozzles need not be positioned in circles. For example, in other embodiments, the micropores may be laid out in other symmetrical geometries, such as a square or star shape. Still further, in other embodiments of the invention, multiple membranes may be used in one nozzle structure, with the micropores in each of the multiple membranes providing parts of the total output spray from the nozzle.

One design parameter for nozzles according to embodiments of the invention is the total number of micropores in the membrane used in the nozzle, i.e., the number of micropores in the nozzle. In some embodiments of the invention, the number of micropores for a spray nozzle range from 40 to 125. Other design parameters of the spray nozzles are the diameter of the micropores in the membrane and the cone angle of the spray emanating from the micropores. In some embodiments of the invention, the diameters of the pores range from about 5 μm to about 10 μm. In more preferred embodiments of the invention, the diameters of the pores range from about 5 μm to about 8 μm. In most preferred embodiment of the invention, the diameters of the pores range from about 5 μm to about 7 μm. Regarding, cone angle, defined by the angle by a spray jet emanating from a micropore through the membrane relative to the axis of the micropore. For example, as shown in the spray nozzle 300 depicted in FIG. 3, some micropores are angled relative to the axis A of the nozzle 300 to emanate at least a portion of the spray at an angle α. In embodiments of the invention, the cone angle of the micropores ranges from about 0° to about 15° in the outer group of micropores. In more preferred embodiments of the invention, the cone angle of the micropores in the outer group ranges from about 5° to about 10°.

Another manner of designing the parameters of spray nozzles according to embodiments of the invention is by looking to the open area in the membrane that is provided by the pores, i.e., the cross-sectional area of a pore on the surface of the membrane multiplied by the number of pores. For example, a membrane in a spray nozzle according to an embodiment of the invention has eighty-two pores having diameters of 6 μm, for a total open area of about 2318 μm2. In embodiments of the invention, the total open area on the surface of the membrane provided by the pores the membranes is from about 1100 μm2 to about 6150 μm2. In preferred embodiments of the invention, the total open area on the surface of the membrane provided by the pores is from about 1100 μm2 to about 3200 μm2. Those skilled in the art will appreciate that the open area for the nozzle will be related to operating conditions for the dispensing systems, particularly the means for generating the force that pushes the formulated product through the membrane. For example, in embodiments of the invention that use a pump mechanism (as will be discussed below), the open area provided by the pores of the membrane will be correlated to the pressure force that the pump generates to cause the formulated product to move through the membrane.

Configurations of nozzles according to specific embodiments of the invention are shown in TABLE 1. The layout of the micropores and membranes for the nozzles shown in TABLE 1 is as shown in FIG. 1. In Design Numbers 4-6, the “inside rings” refer to the inner two concentric rings and the “outside rings” refer to the outer two concentric rings.

TABLE 1

| Design | | | |
|---|---|---|---|
| Number | μPore Diameter | μPore Number | Cone Angle |
| 1 | 6 μm | 41 | 5° |
| 2 | 7 μm | 82 | 10° |
| 3 | 6 μm | 82 | 10° |
| 4 | 5 μm inside rings | 9 inside rings | 0° |
| | 7 μm outside rings | 32 outside rings | 10° |
| 5 | 5 μm inside rings | 18 inside rings | 0° |
| | 7 μm outside rings | 64 outside rings | 10° |
| 6 | 5 μm inside rings | 18 inside rings | 5° inside rings |
| | 7 μm outside rings | 64 outside rings | 15° outside rings |
| 7 | 7 um | 80 | 10° |
| 8 | 7 um | 52 | 10° |

The spray nozzles described herein may be used in conjunction with many different types of dispensing systems. Two types of systems that are well known in the art are automatic aerosol dispensers and base-container aerosol dispensers. A specific example of an automatic aerosol dispensing system is sold under the name GLADE® Automatic Spray by S. C. Johnson & Son, Inc. of Racine, Wisconsin (the assignee of the subject application), and a specific example of a base-container aerosol dispensing system is sold under the name GLADE® Air Freshener by S. C. Johnson & Son, Inc. Example configurations of automatic aerosol dispensers and base-container aerosol dispensers will now be described.

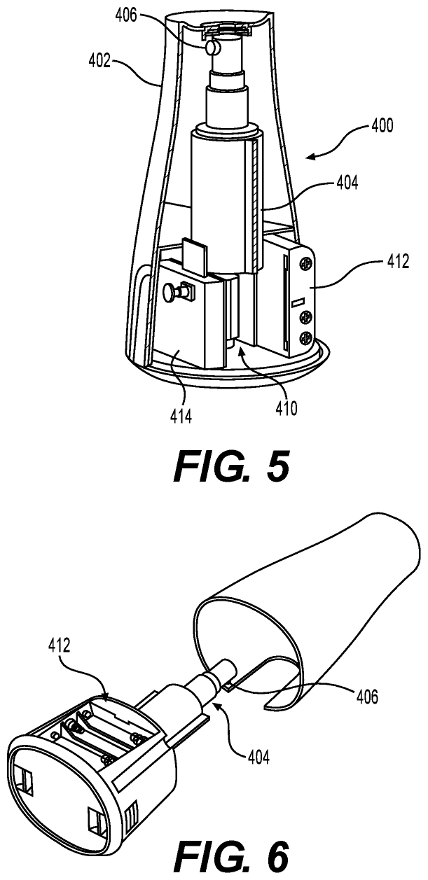

FIGS. 5 and 6 show views of an automatic aerosol dispensing system 400 according to an embodiment of the invention. Such a system provides a metered spray over an extended period of time, such as several weeks. The system 400 includes a housing 402 that encloses the operating parts of the system (part of the housing 402 is removed in FIG. 5 to allow views of the operating parts). The system includes a container 404 for holding the formulated product to be dispensed as the aerosol spray. The container 404 may be replaceable to allow for the system to be refilled when the product from container 404 is used up. A spray nozzle 406 is in fluid communication with the container 404 such that product moves upwardly from the container to the spray nozzle when the system 400 is actuated, and, thus, the product passes through the nozzle 406. The spray nozzle 406 includes a membrane structure with micropores, as described above. An actuating mechanism 410 is provided at the bottom of the container 404. The actuating mechanism 410 is powered by batteries 412 in the depicted embodiment, but in alternative embodiments, the dispensing system may be powered though other means such as an electrical cord and plug. The system 400 also includes a circuit board 414 that has a controller operatively connected to the actuating mechanism 410. Those skilled in the art will easily recognize specific types of containers, actuating systems, and circuit boards that can be used to provide a system 400 as shown in FIGS. 5 and 6.

Unlike prior art automatic aerosol dispensing systems, systems according to embodiments of the invention do not use a propellant gas to pressurize the container as a means for effecting discharge of the product from the container; the product in the container remains at standard atmospheric pressure. Rather than using a propellant gas, in embodiments of the invention the actuating mechanism 410 is a pump. More specifically, the actuating mechanism 410 may be in the form of a positive displacement pump, such as a reciprocating pump or a rotary pump. For example, as a reciprocating pump, the pump can include a piston, plunger, diaphragm, or other structure that functions to provide the force that drives the product out of the container 404. In the case of rotary pumps, the pump may include gears, lobes, screws, vanes, and/or cams to generate the force to discharge the product from the container. In particular embodiments of the invention, the pump can use a precompression valve that functions to provide a highly linear delivery of pressure, which results in a consistent flow of product out of the system.

It should be noted that automatic aerosol dispensing systems according to embodiments of the invention are not limited to the specific configuration shown in FIGS. 5 and 6. In fact, as will be appreciated by a skilled artisan, an automatic system could come in a wide variety of configurations and have many different structures while still generating an aerosol sprays as described herein. For example, while the nozzle in FIG. 5 is positioned to spray out a side of the system, in other embodiments the system could be configured such that the spray is dispensed out the top of the system, i.e., in a vertical direction. Moreover, in still further embodiments the system could be configured with an adjustable the nozzle such that the spray can be dispensed at any angle between horizontal) (0° and vertical) (90°. Examples of configurations of automatic aerosol dispensing systems that may be used in conjunction with the present invention are shown in U.S. Pat. Nos. 8,061,562, 8,678,233, 9,247,724, and 9,833,533, which are hereby incorporated by reference in their entirety.

Because the automatic aerosol dispensing systems according to embodiments of the invention do not use a propellant gas, the systems will not be subject to the regulations associated with systems using VOC-containing propellant gases, such as LPGs. However, as will be demonstrated by the comparison results below, the systems according to the invention may still provide product sprays having equivalent or better properties to sprays generated in LPG systems. Moreover, the sprays from inventive systems are often superior to sprays generated from other types of systems that do not use VOC-containing propellant gases, such as CGAs.

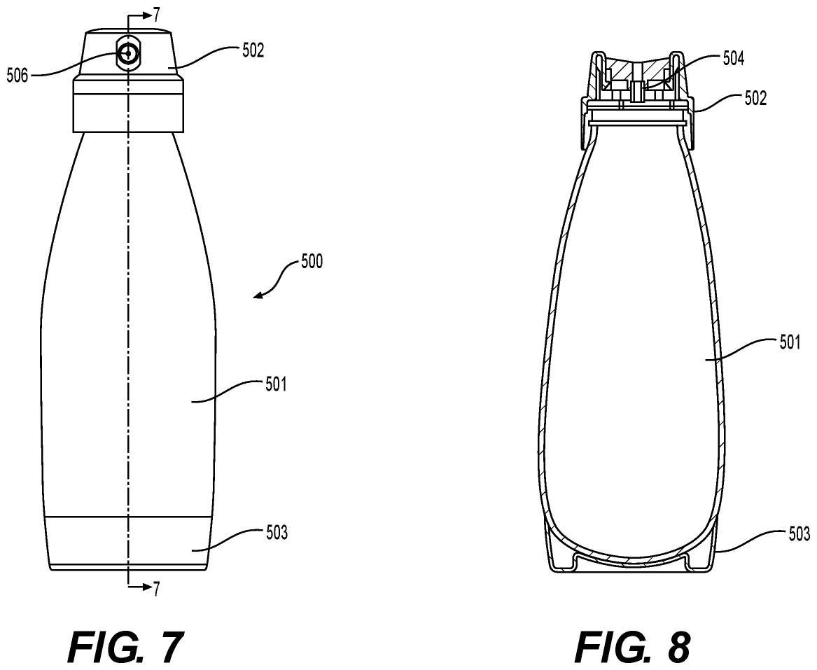

FIGS. 7 and 8 show an example of a base-container aerosol dispensing system 500 according to an embodiment of the invention. The system 500 is designed to be held and actuated on demand by a user. The system 500 includes a bottle 501 that is attached to a base cup 503, with the pressurized product to be dispensed as an aerosol spray being contained in the bottle 501. At the top of the system 500 is a spray mechanism 502 that includes a valve 504 and a spray nozzle 506. As described above, the spray nozzle 506 includes a membrane structure with micropores. The pressurized product contained within the bottle 501 is dispensed through actuation of the spray mechanism 502. Although not shown, a cap may be provided over the spray mechanism 502. Those skilled in the art will recognize the wide variety of valves, spray mechanisms, and caps that could be used with a high-pressure dispensing system of the type described herein.

Base-container aerosol dispensing systems according to embodiments of the invention are not limited to the specific configuration shown in FIGS. 7 and 8. In fact, as will be appreciated by a skilled artisan, the base-container s aerosol dispensing systems could come in a wide variety of configurations and have many different structures while still generating the aerosol sprays as described herein. For example, the base-container aerosol dispensing systems could have configurations as shown in U.S. Pat. Nos. 9,040,024, 9,242,256, 9,393,336, 9,802,752, and 10,633,168, and U.S. Patent Application Pub. No. 2020/0062489, which are hereby incorporated by reference in their entirety.

In embodiments of the invention, a propellant gas is used to effect the discharge of the aerosol product from the base-container dispensing system. For example, the containers in automatic and base-container aerosol dispensing systems as described above may be pressurized with a propellant gas. As discussed above, in many prior art systems, the propellant gas contains a significant amount of volatile organic compounds (VOCs). Propellants can be categorized by being low VOC, no VOC, and VOC exempt. Low VOC propellants, such as dimethyl ether, have a low vapor pressure and are used to reduce the VOC level in products. Dimethyl Ether can be blended with water. Non-VOC propellants include HFC 152a, HFC 134, and Ethane. LPGs such as propane, butane and isobutane are considered VOCs. The propellant HFO 1234ze sold by Honeywell International Inc., of Charlotte, North Carolina, is part of a new generation of VOC exempt propellants. With this, low VOC could be a combination of a VOC propellant with either a VOC-free propellant or solvent or VOC exempt propellant. Base-container aerosol systems according to embodiments of the invention may use low VOC, no VOC, and VOC exempt propellants.

No propellant gas is used in still other embodiments of base-container aerosol systems according to the invention. Instead, in these embodiments a pumping mechanism generates the pressure force to discharge the product through the spray nozzle. Examples of such pumping mechanisms include hand-actuated trigger systems, such as those that are commonly found in consumer products. Specific examples can be seen in U.S. Pat. Nos. 5,474,215, 6,189,739, and 6,708,852, which are hereby incorporated by reference in their entirety. As is the case with the non-propellant automatic aerosol dispensing systems discussed above, the base-container aerosol systems according to the invention do not include VOC-containing LPGs but provide aerosol sprays with desirable properties.

Different formulations are used in a myriad of commercial products to deliver fragrance in the air. Single phase base-container CGA aerosol dispensing systems tend to have water-based formulations that typically include water, emulsifier and fragrance. At other times, water-based formulations are used in conjunction with an LPG to create a dual phase formulation. In such cases, the product is shaken to disperse the LPG within the water formulations. Automatic aerosol dispensing systems often rely on the LPG propellant as a co-solvent for the fragrance. In such automatic devices the fragrance is dissolved in an organic solvent and is blended with the LPG to create a single-phase formulation. Diffusing systems use fragrance oil that usually consist of aroma chemicals mixed with various solvents.

In one embodiment of present invention, an air freshener composition may be a water-based formulation comprising water, emulsifier, and fragrance oil, as follows:

| Ingredient | Weight Percent |
|---|---|
| Water (deionized) | 20-90% |
| Emulsifier | 0-10% |
| Fragrance Oil | trace - 10% |

In other embodiments of the invention, an air freshening compound may be a solvent-based formulation having a co-solvent, such as an alcohol, to facilitate the solubilization of the ingredients. Preferably, the co-solvent is a low molecular weight monohydric C alcohol, such as ethanol, propanol, isopropanol, butanol or isobutanol. Other co-solvents, such as acetone, may also be included in the aerosol composition. In a general embodiment, an emulsifier may be present as set forth above. If the co-solvent is present in the composition in an amount that is insufficient to form an emulsion without the presence of the emulsifier, the emulsifier can be present in such instance in an amount ranging from about 0.4 to about 4 wt. %. Additional adjuvants, such as fragrances, corrosion inhibitors, pH adjustors, antimicrobials, preservatives, and the like, may also be included. Preferred individual ranges for the above-listed adjuvants are from 0 to about 5 wt. %, more preferably from 0 to about 2 wt. %.

In another embodiment, the aerosol composition could consist of just the fragrance oils that are developed by Givaudan Company of Vernier, Switzerland, Takasago International Corporation of Tokyo, Japan, and Surmise AG of Holzminden, Germany. Such products are typically used in plug in scented oils or diffusers. Additionally, the fragrance oil could be added to a solvent such as low vapor pressure solvent such as DPMA (dipropylene glycol ether acetate), Isopar™ (by ExxonMobil Chemical Company of Irving, Texas), DPM (dipropylene glycol monomethyl ether), ethanol, and combinations thereof, can be used to add vapor pressure to enhance the fragrance experience and less fallout. Additionally, low-VOC formulations could be created using low-VOC solvents. For low-VOC formulations, a class of materials includes acetone, dimethyl carbonate, methyl acetate, parachlorobenzotrifluoride (sold under the brand name OXSOL® 100 by Mana of New York, New York), tert-butyl acetate, and propylene carbonate. For solvent-based formulations, the liquid composition may be as follows:

| Ingredient | Weight Percent |
|---|---|
| Fragrance oil | 1-100% |
| Solvent | 0-99% |

In order to demonstrate the unique properties of aerosol sprays according to embodiments of the invention, experiments were conducted to compare the properties of the sprays to sprays dispensed from commercially available aerosol dispensing systems.

To model systems according to embodiments of the invention, inventive systems were created using spray nozzles as described above. In particular, spray nozzles having the configurations of design numbers 1-9 in TABLE 1 above were used in the inventive systems. The spray nozzles were attached to a testing apparatus that simulates the automatic aerosol dispensing systems with pump actuating mechanisms described above. The testing apparatus included a stepper motor driving an actuating plate to generate the force to move the composition out of a container and through a spray nozzle according to embodiments of the invention. The force was comparable to the force that is used to discharge formulated product out of automatic dispensing systems. Both a water-based formulation and a solvent-based formulation (as described above) were tested with the spray nozzles.

The air freshening aerosol sprays dispensed from the testing apparatus were compared to air freshening sprays dispensed from commercially available automatic aerosol spray dispensing systems, base-container aerosol dispensing systems, and other types of aerosol dispensing systems. Five different automatic aerosol dispensing systems were tested. The automatic aerosol dispensing systems used solvent-based formulations. Three of the automatic systems had a configuration as generally found in the automatic systems described above, with the automatic systems using propellant gases to generate the aerosol spray. The other two automatic aerosol systems were wick diffuser systems (hereafter “diffusers”), as are well known in the art. Five different base container aerosol systems having water-based formulations and liquified gas propellants (LPG), and eight different base aerosol systems having water-based formulation and compressed gases (CGA) as propellants were tested. Also tested were two base-container aerosol systems having a bag-on-valve configuration. Such bag-on-valve systems are well-known in the art, and examples of such systems can be seen, for example, in U.S. Pat. No. 9,902,552. Further tested were three trigger-based dispensing systems with water-based formulations, with these dispensing systems having a hand-operated trigger that operates a pump to generate the aerosol spray, as described above.

The aerosol sprays as dispensed from the inventive and comparison systems were evaluated at ambient indoor conditions, i.e., 70° F. and ordinary humidity. The systems were stored for at least twenty-four hours before the tests. The spray rates were determined through weight change during a ten second spray, were reported as grams per second, and are averaged over two sprays during the first forty seconds of sample life. The automatic aerosol dispensing systems were actuated five times for each trial. The actuator for the base-container aerosol systems (i.e., the LPG, CPG, and bag-on-valve systems) were completely depressed for five seconds for each trial, and the systems were shaken appropriately before spraying, allowing up to two to four seconds between shaking and spraying. The trigger-based dispensing systems were actuated five times for each trial. Multiple trials were conducted for each of the systems.

The particle size as mass median diameter, Dv(50), in microns (micrometers, um) was determined from a Malvern laser diffraction particle size analyzer equipped with a 300 mm lens. The base-container aerosol systems and trigger systems were sprayed at a distance of six inches from the beam. The automatic systems were sprayed one to two inches from the lens. All products were sprayed to ensure no products were not on the lenses. A cutoff was applied at 301.7 μm to eliminate ghost peaks caused by “beam steering.” The span factor was determined based on the measurements from the diffraction particle size analyzer and calculated according to the equation described above.

The fallout and product spray distance were tested in a low current room to minimize drifts from air current. Each system was placed on a lab jack and sprayed horizontally on 3 ft×100 in craft paper. Spray height was adjusted to be about eighteen inches from the floor. Each of the systems were sprayed for five seconds or actuated ten times to get reproducible amount of product on the craft paper. The fallout was determined through weight change of the craft paper before and after spraying the product. The weight of the product (package and refill) were determined before and after testing. The amount of product on the paper was determined gravimetrically. The fallout was determined by the quotient of amount of product that accumulated on the craft paper and the amount of product that were sprayed in the air times one hundred:

$$\% Fallout = ( \frac{Weight of Paper After - Weight of Paper Before}{Weight of Product Weight of Product After} ) \times 100$$

The spray distance was determined by visual inspection of the majority of droplets on the paper or in the air. The spray distance was measured (in inched) as the distance from the tip of the product to the end of the plume.

The longevity of the aerosol sprays was measured by monitoring total particle concentration in a twenty-four cubic foot mixed chamber. Equal dosages of the sprays were dispensed into the chamber from the inventive and comparison systems and concentrations were measured using a TSI 3321 aerodynamic particle sizer in twenty second intervals over the course of twenty-five minutes. The inventive and comparison systems were placed upright at the bottom centre of the chamber that is mixed and has no air exchanges. The sampling was done via a one-half inch port approximately two feet above the plume. The longevity was determined as the time to teach a 5% particle concentration.

Results of the experiments are shown in TABLE 2. For the inventive systems listed in the table, “SN” designates the spray nozzle number (per the configurations described above), “WB” indicates that a water-based formulation was used, and “SB” indicates that a solvent-based formulation was used. A discussion of the results follows.

TABLE 2

| | Dv(10) | Dv(50) | Dv(90) | | Distance | |
|---|---|---|---|---|---|---|
| System | (μm) | (μm) | (μm) | SpanFactor | (inch) | % Fallout |
| SN 1 - WB | 25.83 | 39.19 | 59.2 | 0.851 | 46 | 1 |
| SN 2 - WB | 23.46 | 38.41 | 62.21 | 1.009 | 33 | 282 |
| SN 3 - WB | 20.98 | 33.41 | 52.61 | 0.947 | 32 | 173 |
| SN 4 - WB | 28.44 | 42.45 | 63.3 | 0.821 | 36 | 534 |
| SN 5 - WB | 21.14 | 35.36 | 58.49 | 1.057 | 46 | 93 |
| SN 6 - WB | 20.95 | 34.33 | 55.51 | 1.007 | 36 | 1058 |
| SN 7 - WB | 23.32 | 36.42 | 56.53 | 0.91 | 37 | 778 |
| SN 8 - WB | 24.69 | 36.35 | 53.42 | 0.792 | 50 | 74 |
| SN 1 - SB | 25.29 | 38.05 | 57.01 | 0.835 | 28 | 3483 |
| SN 2 - SB | 22.18 | 36.49 | 59.23 | 1.016 | 51 | 639 |
| SN 3 - SB | 21.59 | 33.77 | 52.5 | 0.915 | 39 | 4837 |
| SN 5 - SB | 21.08 | 35.17 | 42.66 | 1.086 | 34 | 4377 |
| SN 6 - SB | 22.12 | 34.73 | 54.08 | 0.922 | 37 | 325 |
| SN 7 - SB | 23.03 | 36.23 | 56.49 | 0.924 | 36 | 444 |
| SN 8 - SB | 26 | 40.45 | 62.36 | 0.899 | 42 | 584 |
| LPG | 17.51 | 34.84 | 65.03 | 1.365 | 94 | 1399 |
| LPG | 17.76 | 36.11 | 68.37 | 1.402 | 94 | 1782 |
| LPG | 18.65 | 42.86 | 85.39 | 1.555 | 84 | 2866 |
| LPG | 21.34 | 51.17 | 101.73 | 1.569 | 94 | 3635 |
| LPG | 14.58 | 29.54 | 54.44 | 1.349 | 98 | 205 |
| Bag-On-Valve | 41.79 | 71.7 | 118.3 | 1.068 | 72 | 5283 |
| Bag-On-Valve | 44.07 | 71.49 | 113.47 | 0.972 | 73 | 5096 |
| CGA | 39.71 | 63.19 | 99.55 | 0.945 | 60 | 656 |
| CGA | 41.77 | 72.73 | 127.33 | 1.171 | 64 | 6979 |
| CGA | 40.04 | 65.31 | 103.89 | 0.976 | 63 | 7276 |
| CGA | 41.81 | 71.53 | 118.57 | 1.072 | 72 | 593 |
| CGA | 43.1 | 75.79 | 129.93 | 1.16 | 60 | 5831 |
| CGA | 37.04 | 66.97 | 114.1 | 1.153 | 56 | 5298 |
| CGA | 36.98 | 64.47 | 107.4 | 1.094 | 58 | 5336 |
| CGA | 39.81 | 67.44 | 111.27 | 1.061 | 58 | 5325 |
| Trigger | 39.94 | 64.9 | 117.63 | 1.492 | 34 | 5114 |
| Trigger | 43.66 | 123.9 | 312.63 | 2.175 | 53 | 7662 |
| Trigger | 38.4 | 65.6 | 110.07 | 1.097 | 43 | 6341 |
| Automatic | 9.65 | 19.59 | 37.82 | 1.476 | 38 | 465 |
| Automatic | 13.22 | 42.05 | 91.77 | 1.87 | 43 | 2227 |
| Automatic | 7.18 | 17.84 | 35.98 | 1.613 | 45 | 214 |
| Diffuser | 3.58 | 7.06 | 13.26 | 1.367 | 20 | 1 |
| Diffuser | 3.59 | 6.84 | 12.47 | 1.318 | 11 | 1 |

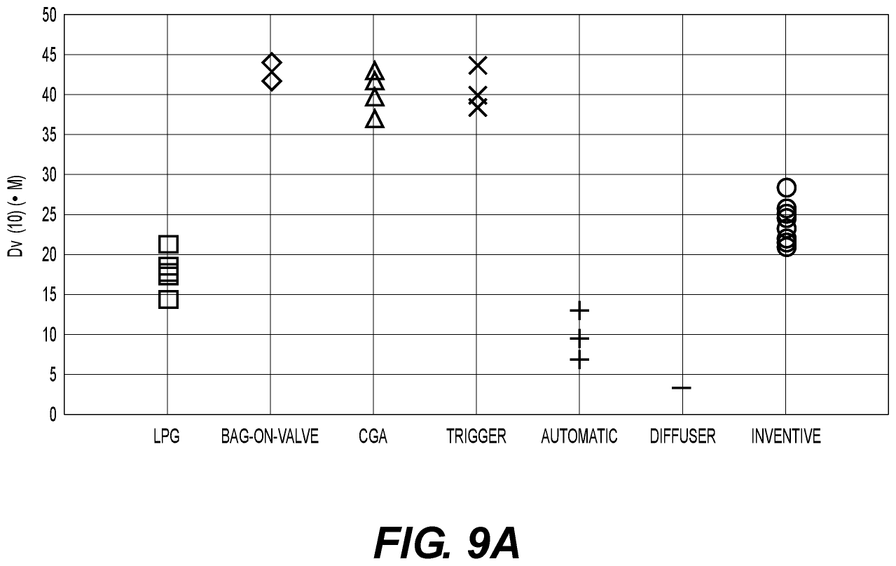

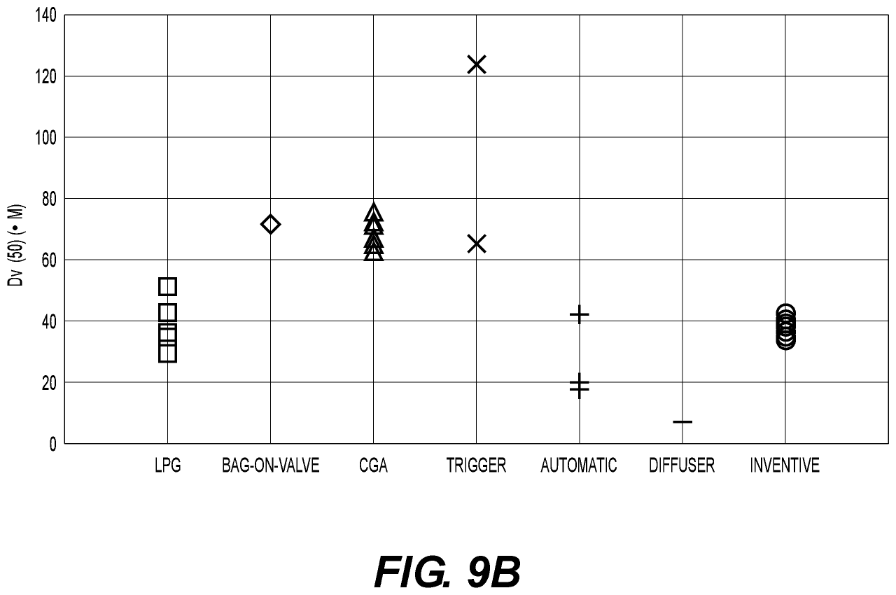

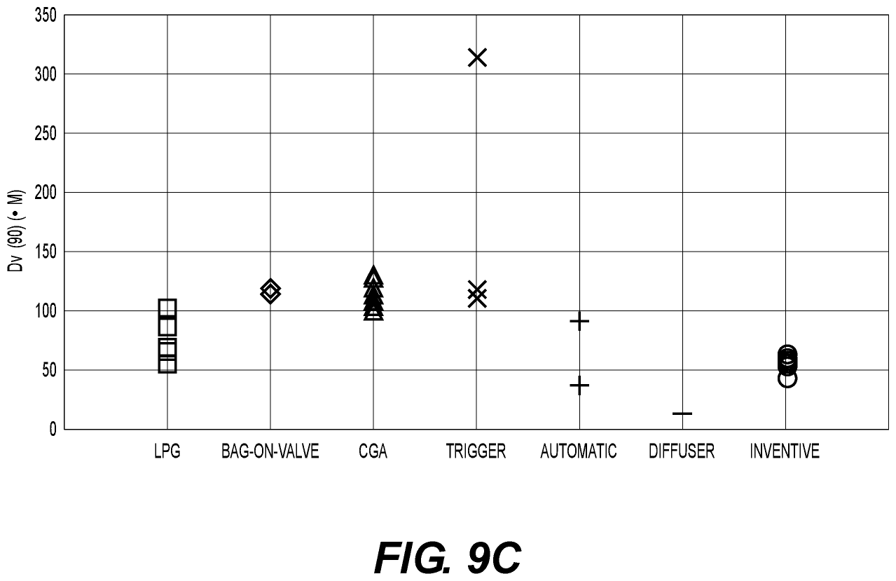

FIGS. 9A-9C shows the particle sizes Dv(10), Dv(50), and Dv(90) of the aerosol sprays dispensed from the inventive systems and the aerosol sprays dispensed from the comparison systems. It can be seen that inventive systems generated particles having Dv(10) sizes from about 21 μm to about 29 μm, Dv(50) sizes in the range of about 35μm to about 42 μm, and Dv(90) sizes in the range of about 43 μm to about 62 μm. It can also be seen that the particle sizes in the inventive aerosol sprays were less than the particle sizes of the CGA and bag-on-valve systems, and that the particle sizes in the inventive aerosol sprays were very comparable to the particle sizes from the LPG dispensing systems. This is significant because, as discussed above, it is desirable to reduce or eliminate the amount of VOCs from the propellants in an aerosol dispensing system. And, unlike the LPG systems, the inventive systems did not use VOC-containing LPG propellants. While the propellants in the CGA and bag-on-valve systems have little or no VOCs, the results show larger particle sizes in the sprays dispensed from such systems. Thus, the inventive aerosol sprays, which were generated without propellants, have particle sizes near that of the particle sizes in aerosol sprays generated with LPG systems and do not have larger particle sizes as found in the sprays from the CGA and bag-on-valve systems.

Table 3 lists a number of alternative nozzle designs according to the invention. The membrane in each of the nozzles of tables 1 and 3 comprises a relatively thin ceramic silicon nitride membrane layer, having a thickness of the order of one micron to a few micron, carried by a rigid silicon support body. In between the nitride layer and the support body is a thin silicon oxide layer. The micropores extend through the membrane layer. The structure is created using state of the art semiconductor technology, including photo-etching and photo-lithography.

The support body comprises cavities underneath the membrane layer to create islands that expose one or more micropores per cavity. The samples that are listed in the table generally comprise a single micropore per island, with the exception of samples 5, 6, 12and 13 that feature double micropores per island, both in the inner rings as well as in the outer rings. The number of micropores per cavity is an additional factor determining the spray density.

TABLE 3

| Design | | | |
|---|---|---|---|
| Number | μPore Diameter | μPore Number | Cone Angle |
| 9 | 6 μm | 41 | 10° |
| 10 | 7 μm | 82 | 20° |
| 11 | 5 μm inside rings | 9 inside rings | 0° |
| | 7 μm outside rings | 32 outside rings | 20° |
| 12 | 5 μm inside rings | 18 inside rings | 0° |
| | 7 μm outside rings | 64 outside rings | 20° |
| 13 | 7 μm inside rings | 18 inside rings | 10° |
| | 5 μm outside rings | 64 outside rings | 30° |
| 14 | 7 μm inside rings | 9 inside rings | 0° |
| | 5 μm outside rings | 32 outside rings | 10° |
| 15 | 7 μm | 80 | 20° |
| 16 | 7 μm | 52 | 20° |

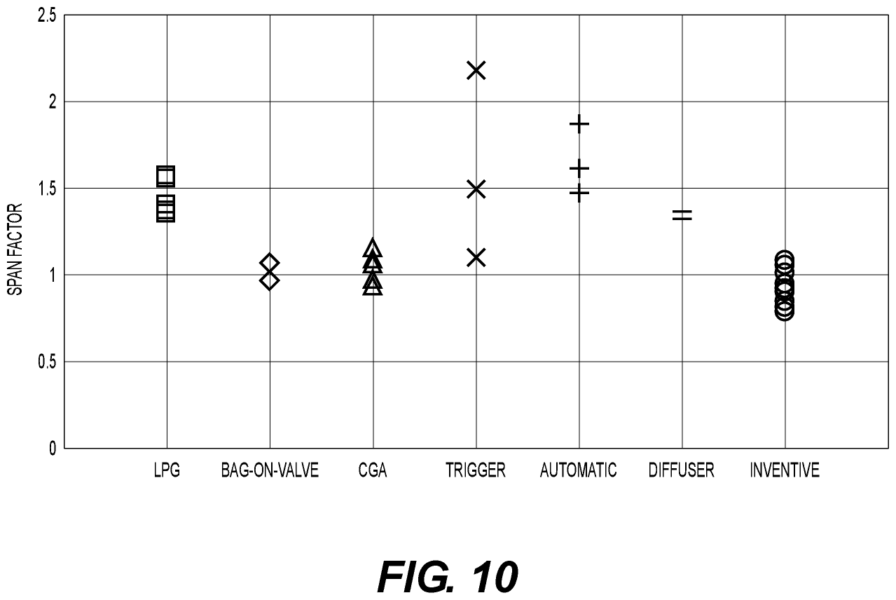

FIG. 10 shows the span factors for particles of the aerosol sprays dispensed from the inventive systems and the span factors for the particles dispensed from the comparison systems. The data shows that the inventive systems had span factors that were just as low, if not lower than the comparative systems. As generally discussed above, a lower span factor means that the particles in the sprays are more consistent in size. And, for air freshening products, consist particle size provides for a more consistent consumer fragrance experience.

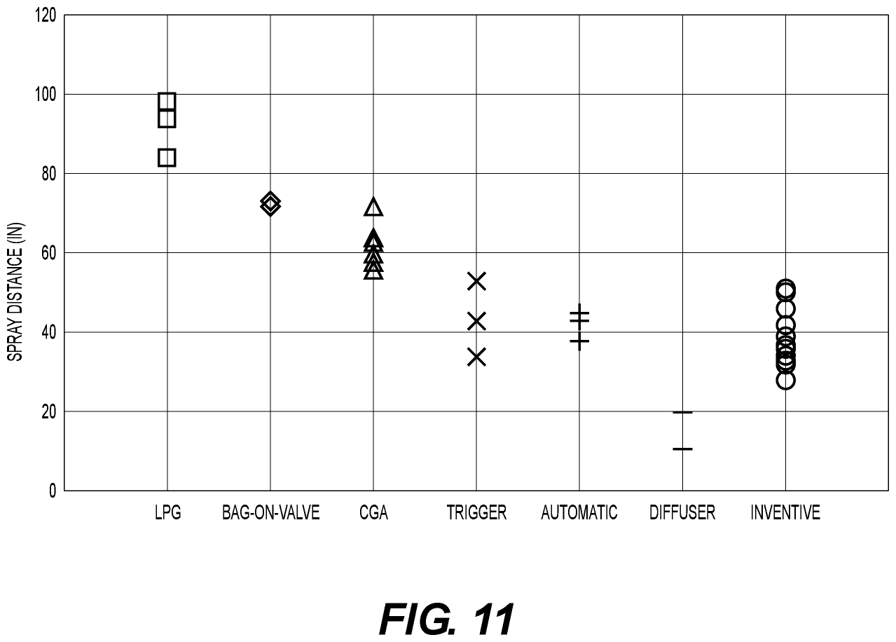

FIG. 11 shows the spray distances for particles of the aerosol sprays dispensed from the inventive systems and the spray distances for particles dispensed from the comparison systems. The spray distances from the inventive systems were generally comparable to the distances from the comparison systems, particularly the automatic and trigger-based systems.

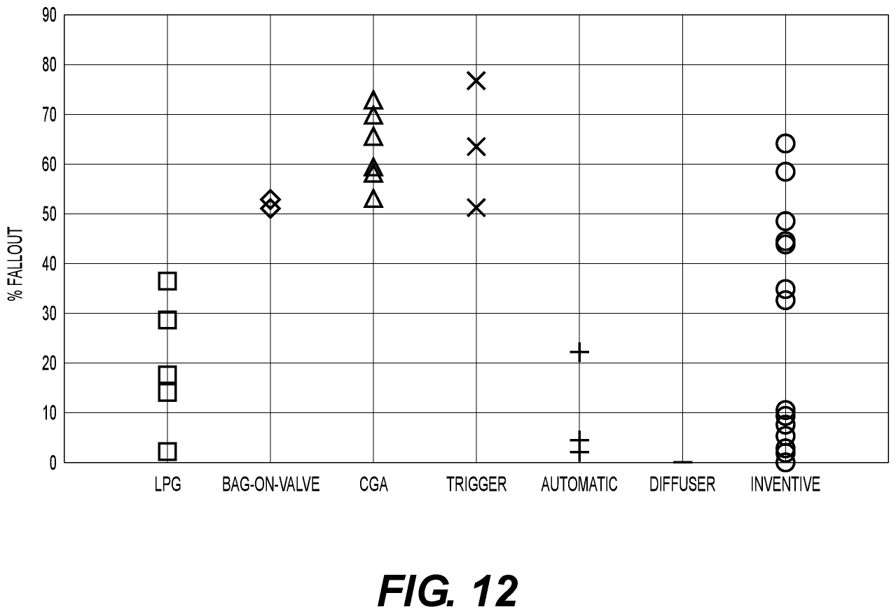

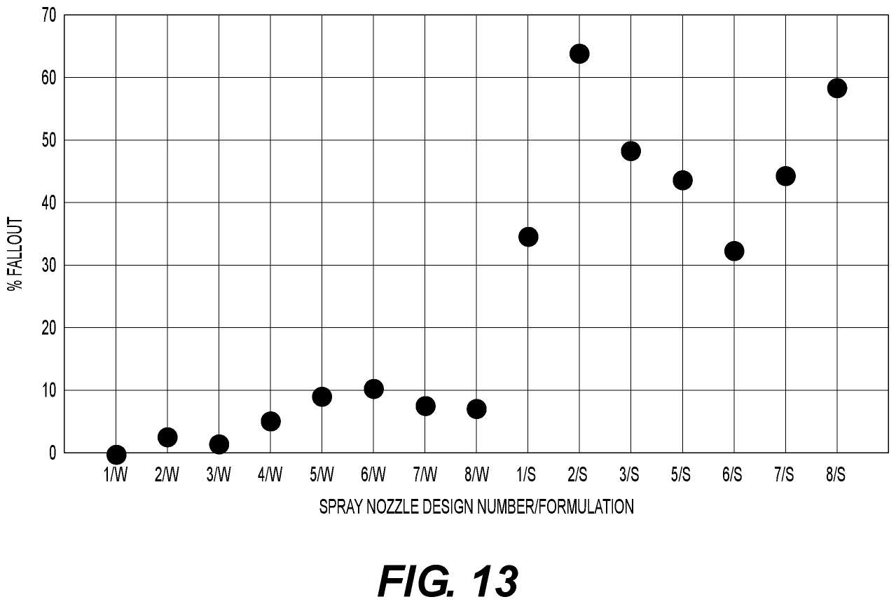

FIG. 12 shows a comparison of the fallout for particles of the aerosol sprays dispensed from the inventive systems and comparison systems. FIG. 13 shows a further comparison of the fallout generated with different spray nozzles and formulations according to embodiments of the invention. In particular, the Spray Nozzle

Design Numbers 1-8 were tested with the water-based formulation (designated as 1/W, 2/W, etc. in FIG. 13) and Spray Nozzle Design Numbers 1, 2, and 4-8 were tested with the solvent-based formulation (designated as 1/S, 2/S, etc. in FIG. 13).

Fallout is a significant factor in the evaluation of aerosol sprays because too much fallout is undesirable from a consumer satisfaction standpoint. The data shown in FIGS. 12 and 13 demonstrates that the inventive aerosol sprays with the water-based formulation had fallouts ranging from about zero to 10%. Notably, this range of fallout was less than the fallout from the CGA and bag-on-valve systems, and on par with the fallout from the LPG base-container systems. As discussed above, the inventive aerosol sprays were advantageous in comparison to the LPG propellant base-container systems because inventive systems do not include propellant gases. Thus, the inventive systems having the water-based formulations had the upside of low fallout found in LPG systems without the downside of high-VOC propellants that are found in LPG systems.

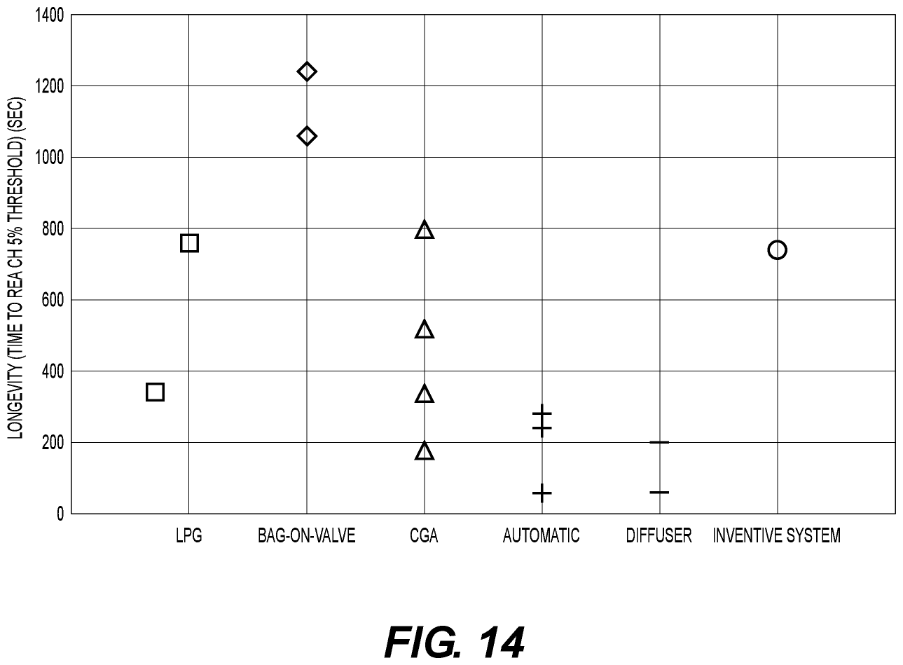

The results of the longevity tests for an inventive system and comparison systems are shown in TABLE 4 and FIG. 14. As discussed above, the longevity was determined as the time to teach a 5% particle concentration. For these tests, a spray nozzle with Design Number 1 and a water-based formulation was used. For comparison, one LPG system, two bag-on-valve systems, four CGA systems, two automatic systems, and two diffuser systems were tested.

TABLE 4

| System | Longevity |
|---|---|
| Inventive | 740 |
| LPG | 760 |
| Bag-on-Valve | 1060 |
| Bag-on-Valve | 1240 |
| CGA | 340 |
| CGA | 180 |
| CGA | 800 |
| CGA | 520 |
| Automatic | 60 |
| Automatic | 280 |
| Automatic | 240 |
| Diffuser | 200 |
| Diffuser | 60 |

The longevity of the aerosol spray from the inventive systems was greater than the longevity of the aerosol sprays from the automatic and diffuser systems, greater than most of the CGA propellant systems, and comparable to the LPG propellant system.

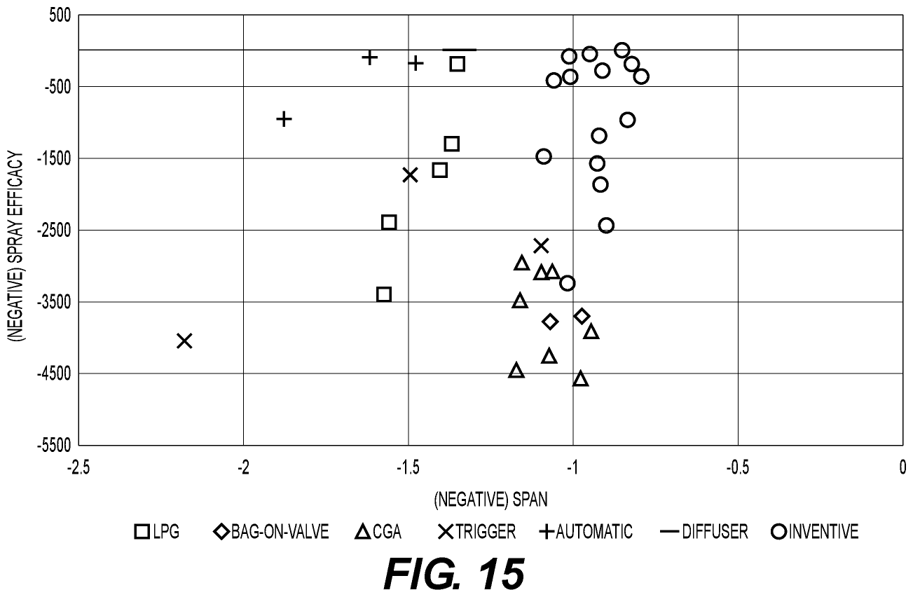

FIG. 15 shows the spray experience factors of aerosol sprays from the inventive systems and the spray experience factors of the aerosol sprays from the comparison systems. As described above, the spray experience factor is defined by the combination of spray efficacy and particle span, where spray efficacy is defined as the negative of the product of the percent fallout and spray distance and the span is defined as the negative of the span factor of the particles. As the results in FIG. 15 show, the inventive aerosol sprays had spray experience factors with a spray efficacy of about 0 to about −3300 and a (negative) span of about −0.75 to about−1.1. None of the comparative systems had a spray experience factors in the range of the inventive systems. This demonstrates that the inventive aerosol products will perform better when used, for example, as an air freshening product as the product will have a combination of properties including more consistent particle size (low span factor) and greater spray efficacy (more spray distance and less fallout).

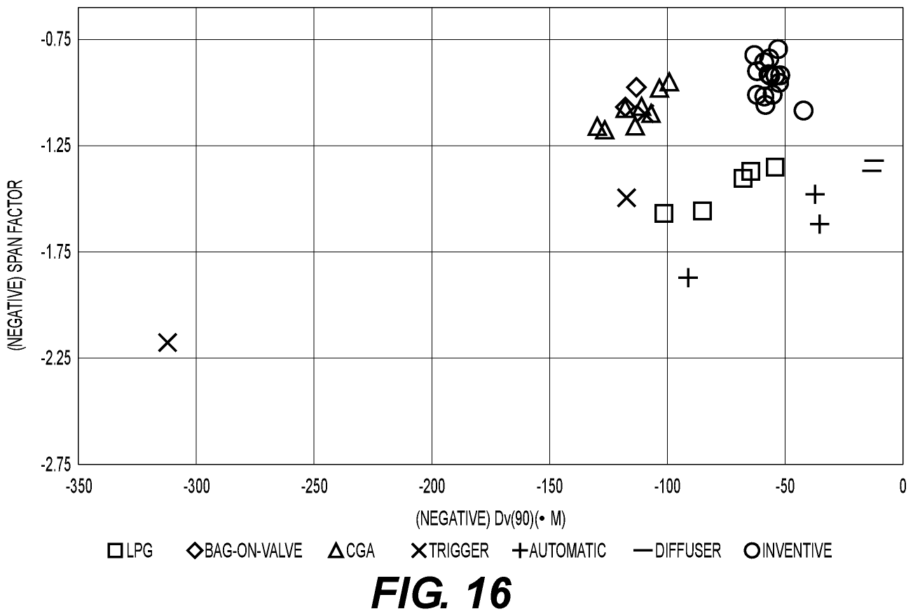

FIG. 16 shows particle quality factor for the inventive systems and the comparison systems. As discussed above, the particle quality factor is defined by the negative Dv(90) particle size for the spray and the negative of the span factor for the spray particles. The results shown in FIG. 16 demonstrate that the inventive aerosol sprays have particle quality factors not found in the sprays from the comparison systems, with the (negative) Dv(90) ranging from about −40 μm to about −65 μm and the (negative) span factor ranging from about −0.75 to about −1.1. Thus, the sprays from the inventive systems contained a lower number of large sized particles that fell within a narrow range. This particle quality for the inventive sprays provides for outstanding air freshening products.

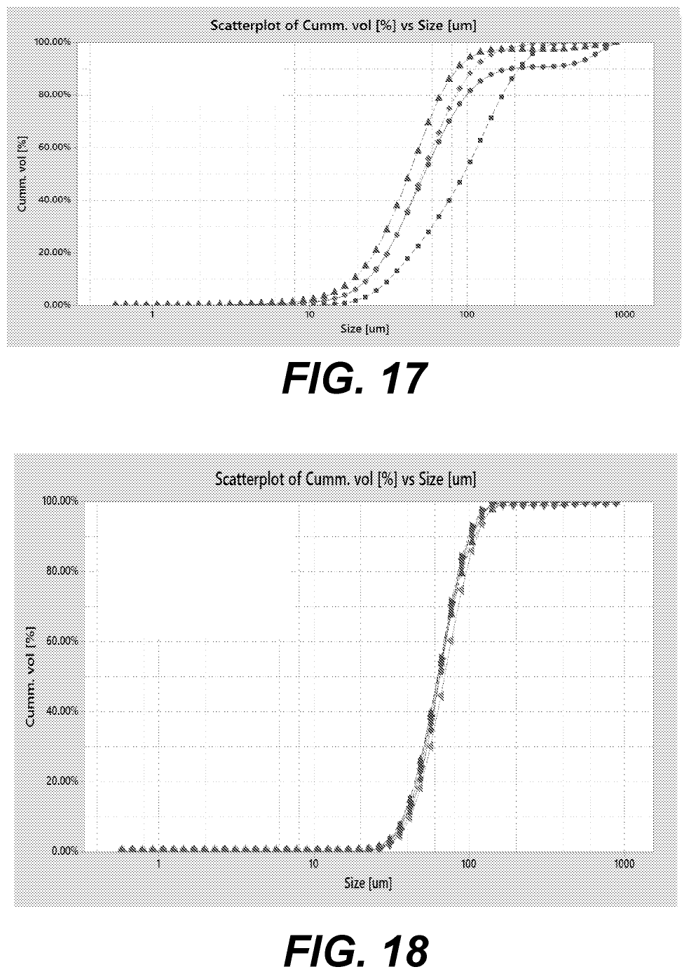

FIG. 17 shows the particle size distribution of particles in a conventional spray created by a conventional nozzle design for several liquid compositions, including varying liquid compositions other than fragrances. FIG. 18 shows the particle size distribution withing an aerosol according to the invention, using the method and dispensing system of the invention of the same liquid compositions. The nozzles used comprise micropores that each release a Rayleigh type spray jet. Clearly the invention (FIG. 18) delivers a significantly sharper particle (droplet) size distribution than the conventional nozzle of FIG. 17. The aerosol of FIG. 18 is particularly almost void of any droplets smaller than 15 μm or larger than little over 100 μm.

As described herein, the present invention provides aerosol sprays, methods of generating aerosol sprays, and systems for generating aerosol sprays that are not found in the prior art. The inventive aerosol sprays have properties and combinations of properties that make the sprays ideal for many applications, particularly as air freshening products.

While various embodiments of the invention have been described above, it should be understood that they have been presented by way of example, and not limitation. It is apparent to persons skilled in the relevant arts that various changes in form and detail can be made therein. Thus, the invention should not be limited by any of the above-described embodiments, but should be defined only in accordance with the following claims and their equivalents.

1. An aerosol spray dispensing system comprising:
a non-pressurized container containing a liquid formulation;
a spray nozzle in fluid communication with the container, the spray nozzle including a membrane having micropores through which the product passes as the product is dispensed from the system; and a pump configured to provide a force causing the liquid formulation to move from the container and through the spray nozzle such that the liquid formulation is discharged from the system as an aerosol spray, wherein the micropores comprise a first group of at least one micropore adjacent a second group of micropores, wherein said first group releases a first aerosol spray portion, wherein the second group releases a second aerosol spray portion, adjacent the first aerosol portion, and wherein a spray density of the first aerosol portion emanating from the first group is larger than a spray density of the second aerosol portion emanating from the second group.

2. The aerosol spray dispensing system according to claim 1, wherein the first group comprises micropores having a larger pore density than a pore density of the micropores within the second group of micropores.

3. The aerosol spray dispensing system according to claim 1, wherein the first group comprises micropores having a larger average size than an average size of the micropores within the second group of micropores.

4. The aerosol spray dispensing system according to claim 1, wherein the micropores within the second group of micropores release a microjet under a diverging jet angle with respect to an axis of the spray nozzle.

5. The aerosol spray dispensing system according to claim 4, wherein the at least one micropore within the first group releases a microjet substantially parallel to the axis of the spray nozzle.

6. The aerosol spray dispensing system according to claim 1, wherein the second group of micropores surrounds the first group.

7. The aerosol spray dispensing system according to claim 6, wherein the first group and the second group each comprise at least one ring of micropores, said rings of micropores being substantially con-centric with one another.

8. The aerosol spray dispensing system according to claim 1, wherein particles in the spray have a Dv(50) size of about 30 pm to about 70 pm.

9. The aerosol spray dispensing system according to claim 1, wherein the aerosol spray emanates from the micropores of the membrane in the spray nozzle in Rayleigh jets that subsequently break up particles of the aerosol spray.

10. The aerosol spray dispensing system according to claim 1, wherein the nozzle includes 40 to 125 micropores, and wherein the micropores range in diameter from about 5 pm to about 10 pm.

11. The aerosol spray dispensing system according to claim 1, wherein micropores range in diameter from about 5 pm to about 8 pm.

12. The aerosol spray dispensing system according to claim 1, wherein a total open area on the surface of the membrane provided by the pores is from about 1100 pm2 to about 6150 pm2.

13. The aerosol spray dispensing system according to claim 12, wherein a total open area on the surface of the membrane provided by the pores is from about 1100 pm2 to about 3200 pm2.

14. The aerosol spray dispensing system according to claim 1, wherein the liquid compound includes one or more of an air freshening composition including (i) fragrance oil and (ii) water or a solvent; a deodorizer agent; a disinfectant agent; an insecticide agent; and a cleaning agent.

15. The aerosol spray dispensing system according to claim 1, wherein the pump is a positive displacement pump.

16. A method of generating an aerosol spray, the method comprising:
forcing a liquid composition out of a non-pressurized container; and passing the liquid composition through micropores on a membrane such that
Rayleigh jets are produced, with the Rayleigh jets subsequently breaking up into particles of the aerosol spray, wherein the aerosol spray comprises a first aerosol portion and a second aerosol spray portion, adjacent the first aerosol portion, and wherein a spray density of the first aerosol portion is larger than a spray density of the second aerosol portion.

17. The method according to claim 16, wherein the second aerosol portion substantially forms a cone having a diverging cone angle with respect to an axis of the spray nozzle.

18. The method according to claim 17, wherein the first aerosol portion propagates substantially parallel to the axis of the spray nozzle.

19. The method according to claim 16, wherein the second aerosol portion surrounds the first aerosol portion.

20. The method according to claim 19, wherein the first aerosol portion and the second aerosol portion each comprise at least one aerosol ring, said aerosol rings being substantially concentric with one another.

21. The method according to claim 16, wherein the liquid composition includes one or more of an air freshening composition including (i) fragrance oil and (ii) water or a solvent; a deodorizer agent; a disinfectant agent; an insecticide agent; and a cleaning agent.

22. The method according to claim 16, wherein the micropores range in diameter from about 5 pm to about 10 pm.

23. The method according to claim 21, wherein the micropores range in diameter from about 5 pm to about 8 pm.

24. The method according to claim 16, wherein the aerosol spray has a spray experience factor with a (negative) spray efficacy of up to about −3300 and a (negative) span factor of about up to about −1.25.

25. The method according to claim 16, wherein the aerosol spray has a particle quality factor with a (negative) Dv(90) particle size of up to about −90 pm and a (negative) span factor of up to about −1.25.

26. The method according to claim 16, wherein a total open area on the surface of the membrane provided by the pores is from about 1100 pm2 to about 6150 pm2.

27. The method according to claim 16, wherein a total open area on the surface of the membrane provided by the pores is from about 1100 pm2 to about 3200 pm2.

28. An aerosol spray comprising particles of a liquid composition, wherein the aerosol comprises a first aerosol portion adjacent an second aerosol portion, said first aerosol portion having a larger spray density than said second aerosol portion, wherein the particles have a Dv(50) particle size of about 30 pm to about 70 pm, and wherein the aerosol spray has a spray experience factor with a (negative) spray efficacy of about 0 to about −3300 and a (negative) span factor of up to about −1.25.

29. The aerosol spray according to claim 27, wherein the liquid composition comprises one or more of an air freshening composition including (i) fragrance oil and (ii) water or a solvent; a deodorizer agent; a disinfectant agent; an insecticide agent; and a cleaning agent.

30. The aerosol spray according to claim 1, wherein the particles have a Dv(50) particle size of about 35 pm to about 45 pm.

31. The aerosol spray according to claim 27, wherein the composition includes a fragrance oil and water, and wherein the aerosol spray has a spray experience factor with a (negative) spray efficacy of about 0 to about −400 and a (negative) span factor of about −0.75 to about −1.0.

32. The aerosol spray according to claim 16, wherein the composition includes a fragrance oil and a solvent, and wherein the aerosol spray has a spray experience factor with a (negative) spray efficacy of about −950 to about −3300 and a (negative) span factor of about −0.8 to about −1.1.

33. An aerosol spray comprising particles of a liquid composition including a fragrance oil, wherein the particles have a Dv(50) particle size of about 30 pm to about 70 pm, and wherein the aerosol spray has a particle quality factor with a (negative) Dv(90) particle size of up to about −90 pm and a (negative) span factor of up to about −1.25.

34. The aerosol spray according to claim 32, wherein the particles have a Dv(50) particle size of about 35 pm to about 45 pm.

35. The aerosol spray according to claim 32, wherein the composition includes water, and wherein aerosol spray has a particle quality factor with a (negative) Dv(90) particle size of about −50 pm to about −65 pm and a (negative) span factor of −0.75 to about −1.0.

35. The aerosol spray according to claim 32, wherein the composition includes a solvent, and wherein aerosol spray has a particle quality factor with a (negative) Dv(90) particle size of about −40 pm to about −65 pm and a (negative) span factor of −0.8 to about −1.1.
